(19) United States
(12) Patent Application Publication
(10) Pub. No.: US 2024/0247051 A1
(43) Pub. Date: Jul. 25, 2024

(54) METHODS OF IMMUNOTHERAPY

(71) Applicant: Yigal Adir, North Hollywood, CA (US)

(72) Inventor: Yigal Adir, North Hollywood, CA (US)

(21) Appl. No.: 18/430,151

(22) Filed: Feb. 1, 2024

Related U.S. Application Data
(63) Continuation-in-part of application No. 16/873,472, filed on Apr. 17, 2020.

(51) Int. Cl. C07K 16/08; A61K 39/00; C07K 16/12; C07K 16/14; C07K 16/20

(52) U.S. Cl. CPC C07K 16/08; C07K 16/12; C07K 16/14; C07K 16/20; A61K 2039/505; C07K 2317/10; C07K 2317/20

(57) ABSTRACT

The disclosure features a method which includes isolating an infectious agent from a human patient infected with the infectious agent, obtaining one or more immunoproteins, immunopolypeptides, and immunopeptides from serum from a non-domesticated wild animal by inoculating, in vivo, the non-domesticated wild animal with the isolated infectious agent or inoculating, in vitro, blood taken from the non-domesticated wild animal with the isolated infectious agent, comparing a protein, polypeptide, and peptide composition of serum obtained from blood from the non-domesticated wild animal inoculated in vivo or serum obtained from in vitro inoculated blood from the non-domesticated wild animal to that of a serum sample obtained from blood from the non-domesticated wild animal prior to inoculation or from non-inoculated blood, to identify the one or more immunoproteins, immunopolypeptides, and immunopeptides, and isolating and purifying the one or more immunoproteins, immunopolypeptides, and immunopeptides, and administering them to the infected human patient.

### CROSS-REFERENCE TO RELATED APPLICATIONS

The present application is a Continuation-in-Part (CIP) application of U.S. patent application Ser. No. 16/873,472, filed on Apr. 17, 2020, published as U.S. Patent Application Publication No. US 2021/0322539 A1 on Oct. 21, 2021, the disclosure of which is hereby incorporated by reference in its entirety.

### FIELD

The present disclosure is directed to the fields of immunology and biologics. More particularly, the present disclosure is directed to immunotherapeutic biologics derived from non-domesticated wild animals as host animals inoculated with an infectious agent isolated from a human patient, as well as methods of treating human patients suffering from an infection caused by the infectious agent with the immunotherapeutic biologics.

### BACKGROUND

Infectious disease outbreaks often occur without warning. Examples of infectious disease outbreaks include HIV, Zika, Marburg, SARS, MERS, and SARS-COV-2. An infectious disease outbreak can be epidemic or pandemic based on the population effected. Very often, treatments for epidemic or pandemic infections are ineffective or lacking, particularly when the infectious agent has only recently infected humans or is genetically divergent from known infectious agents. Epidemic or pandemic infections can quickly overwhelm hospital capacity, decimate the most vulnerable members of society such as the elderly and immunocompromised, and cost society billions of dollars from mitigation measures such as lockdowns. As such, there is a great need for immediately effective treatments for such infectious disease outbreaks.

### SUMMARY

In general, the disclosure features a method. The method includes isolating an infectious agent from a human patient infected with the infectious agent, obtaining one or more immunoproteins, immunopolypeptides, and immunopeptides from serum from a non-domesticated wild animal by inoculating, in vivo, the non-domesticated wild animal with the isolated infectious agent or inoculating, in vitro, blood taken from the non-domesticated wild animal with the isolated infectious agent, comparing a protein, polypeptide, and peptide composition of serum obtained from blood from the non-domesticated wild animal inoculated in vivo or serum obtained from in vitro inoculated blood from the non-domesticated wild animal to that of a serum sample obtained from blood from the non-domesticated wild animal prior to inoculation or from non-inoculated blood, to identify the one or more immunoproteins, immunopolypeptides, and immunopeptides, and isolating and purifying the one or more immunoproteins, immunopolypeptides, and immunopeptides, and administering the one or more immunoproteins, immunopolypeptides, and immunopeptides to the human patient infected with the infectious agent.

Features of the method can include the following. The infectious agent can be a virus, bacteria, fungus, helminth, protozoa, or prion. The one or more immunoproteins, immunopolypeptides, and immunopeptides can be chosen from antibodies, cytokines, components of the complement system, and acute phase proteins. The method can further include manufacturing the one or more immunoproteins, immunopolypeptides, and immunopeptides in a biologics facility before administration to the human patient, which can include engineering the one or more immunoproteins, immunopolypeptides, and immunopeptides during manufacturing to increase stability, increase efficacy, increase solubility, increase protease resistance, reduce immunogenicity, and/or improve pharmacokinetic parameters of the immunoproteins, immunopolypeptides, and immunopeptides in humans. The non-domesticated wild animal can be a hippopotamus, snow leopard, amur leopard, puma, dogfish shark, dolphin, zebra, buffalo, ostrich, elephant, alligator, crocodile, hyena, or bear. The non-domesticated wild animal can be a scavenger, such as a crow, vulture, condor, hawk, eagle, hyena, jackal, leopard, lion, opossum, raccoon, raven, seagull, lobster, crab, shark, Tasmanian devil, bear, coyote, wolf, Komodo dragon, crocodile, and alligator.

It should be understood that the above method is not to be considered a limitation on the invention defined by the claims. The featured method can be implemented in one or more ways using one or more features depicted in the drawings, described in the detailed description, and set forth in the claims.

### BRIEF DESCRIPTION OF THE DRAWINGS

The accompanying drawings illustrate certain aspects and principles of the implementations set forth, and should not be construed as limiting.

FIG. 1 is a drawing depicting an implementation of the method of the disclosure wherein the non-domesticated wild animal is a hippopotamus.

FIG. 2 is a drawing depicting an implementation of the method of the disclosure wherein the non-domesticated wild animal is a crocodile.

### DETAILED DESCRIPTION

Reference will now be made in detail to various illustrative implementations. It is to be understood that the following discussion of the implementations is not intended to be limiting.

The implementations of the present disclosure employ, unless otherwise indicated, conventional techniques of molecular biology (including recombinant techniques), microbiology, virology, cell biology, biochemistry, nucleic acid chemistry, immunology, and pharmacy; which are well known to those skilled in the art. Such techniques are explained fully in the literature, such as, Adejare A, ed. (2020), Remington: The Science and Practice of Pharmacy, 23^{rd} Ed. (Academic Press, Cambridge, MA), hereafter Remington; Analytical Methods Committee Amctb No. (2013) PCR—the polymerase chain reaction. Anal Methods. December 19; 6(2):333-336; Ausubel F M et al. (2003). Current Protocols in Molecular Biology (John Wiley & Sons, Hoboken, NJ); Baer A, Kehn-Hall K (2014). Viral concentration determination through plaque assays: using traditional and novel overlay systems. J Vis Exp. November 4(93):e52065; Cassedy A, Parle-McDermott A, O'Kennedy R. (2021) Virus Detection: A Review of the Current and Emerging Molecular and Immunological Methods. Front Mol Biosci. April 20; 8:637559; Current Protocols in Nucleic Acid Chemistry (2017) (John Wiley & Sons, Inc., NY); Current Protocols in Protein Science (2017) (John Wiley & Sons, Inc., NY); Cutler, P, ed. (2004). Protein Purification Protocols (Humana Press, Totowa, NJ); de St Groth, S. E, and Scheidegger, D. (1980). Production of monoclonal antibodies: Strategy and tactics. J. Immunol. Methods 35, 1-21; Donohoe, P. J., Macardle, P. J., and Zola, H. ed. (1994). Making and using conventional mouse monoclonal antibodies. In “Monoclonal Antibodies: The Second Generation”, Biological Sciences, (Coronet Books, Philadelphia, PA); Dulbecco R, Vogt M. (1953). Some problems of animal virology as studied by the plaque technique. Cold Spring Harb Symp Quant Biol. 18:273-9; Freshney, R. I. (2002). “Culture of Animal Cells: A Manual of Basic Technique,” 4th Ed. Wiley-Liss, New York; Goding, J. W. (1986). “Monoclonal Antibodies: Principles and Practice,” 2nd Ed. (Academic Press, London); Green, M. R. and Sambrook, J (2012). Molecular Cloning: A Laboratory Manual, 4^{th} Ed. (Cold Spring Harbor Laboratory Press, Cold Spring Harbor, N.Y.); Greenfield, E., ed. (2012). Antibodies: A Laboratory Manual, 2^{nd} Ed. (Cold Spring Harbor Laboratory Press, Cold Spring Harbor, N.Y.); Harlow E. and Lane D. (1999) Using Antibodies: A Laboratory Manual (Cold Spring Harbor Laboratory Press, Cold Spring Harbor, N.Y); Harrison, M., Roe, I. E., Harris, A eds. (2003). General Techniques of Cell Culture. In Handbooks in Practical Animal Cell Biology (Cambridge Univ. Press, Cambridge, U.K); Leenaars et al. (2005). Critical Steps in the Production of Polyclonal and Monoclonal Antibodies: Evaluation and Recommendations. 46(3):269-279; Lodish, H. et al. (2021) Molecular Cell Biology, (W.H. Freeman and Company, NY); Mullis, K. B., Ferré, F. and Gibbs, R. A., eds. (1994). The polymerase chain reaction (PCR). (Birkhäuser Verlag A G, Basel, Switzerland); Walker, J. M., ed. (2009) The Protein Protocols Handbook (Humana Press, Totowa, NJ). These publications are each individually incorporated by reference herein in their entireties.

Unless otherwise defined, all terms of art, notations and other scientific terminology used herein are intended to have the meanings commonly understood by those of skill in the art to which this invention pertains.

Described herein are methods of immunotherapy that derive antibodies and other disease fighting components from wild animals rather than domesticated animals. In one implementation, a bacteria, virus, or other infectious agent is isolated from a patient with an infectious disease. The isolated bacteria, virus, or other infectious agent is then administered in vivo to a non-domesticated wild animal obtained from its natural habitat. In other implementations, blood is taken from the non-domesticated wild animal and cultured and then exposed in vitro to the isolated bacteria, virus, or other infectious agent. The non-domesticated wild animal can be one that lives in harsh environments and relies on scavenging carrion for food sources, such as a vulture or hyena. Unlike domesticated or lab-based breeds of animals, the present inventor has theorized that non-domesticated wild animals have evolved to have more sophisticated and enhanced immune systems given their environments and eating habits.

Once the non-domesticated wild animal is administered the bacteria, virus, or other infectious agent in vivo, their blood is collected after a period of time (or, alternatively, samples of cultured blood taken from the non-domesticated wild animal and exposed to the isolated bacteria, virus, or other infectious agent in vitro are taken). Their infected or exposed blood or serum is compared to their normal or non-exposed blood or serum, such as blood or serum taken before inoculation or non-inoculated blood or serum, and the components that are different (e.g., soluble proteinaceous disease fighting components such as immunoglobulins and cytokines) are identified through standard laboratory techniques. The identified immunotherapeutic proteinaceous effectors or agents including antibodies, cytokines such as interleukins, interferons, chemokines, lymphokines, monokines, tumor necrosis factors, transforming growth factors, and haematopoietins, components of the complement system, and acute phase proteins are isolated from blood or serum samples taken from the in vivo exposed animal or the in vitro exposed blood and then purified. The antibodies and other disease fighting components are then either administered directly to the human patient with the infectious disease, or the composition of the disease fighting components is determined in a laboratory and then generated, engineered and/or manufactured in large scale production of a biologic or biologics or pharmaceuticals and administered to patient populations suffering from the infectious disease.

The administered antibodies or other disease fighting components can be supplemented with additional components to assist in the healing process such as vitamins, medicines, supplements, other antibodies, enzymes to prevent blood clots, or antivirals or antibiotics that can slow the proliferation of the infectious agent as the patient's immune system and administered components are able to take effect.

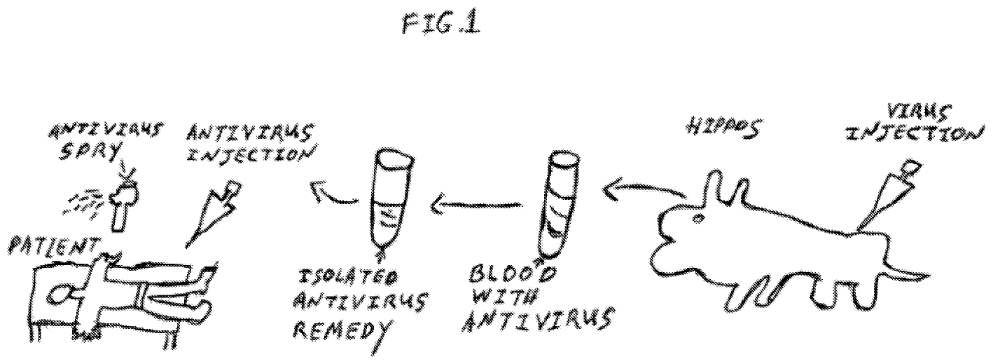

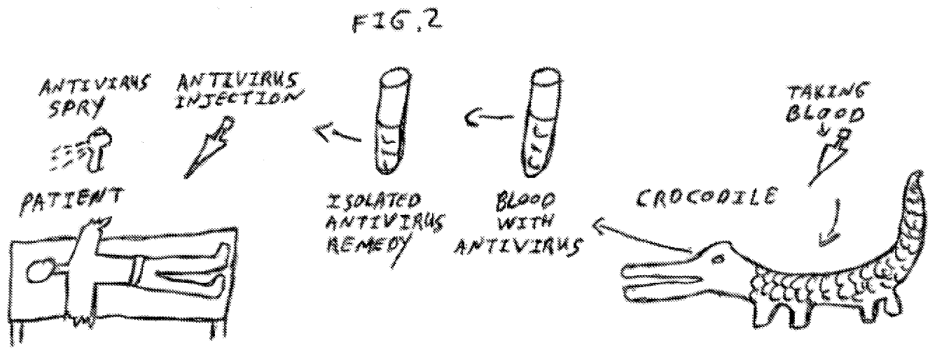

Some non-limiting examples of non-domesticated wild animals that the bacteria, virus, or other infectious agent can be administered to, either in vivo or to their blood samples in vitro, include a range of wild animal species such as hippos, snow leopards, amur leopards, puma, dogfish sharks, dolphins, zebras, buffalos, ostriches (can also include ostrich eggs), elephants, alligators, crocodiles, hyenas, and bears. The non-domesticated wild animal is one that does not suffer pathological effects from the bacteria, virus, or other infectious agent isolated from a human patient or patients due to its superior immune system that can handle a broader range of infectious agents than a human's immune system. In one implementation, the non-domesticated wild animal that the bacteria, virus, or other infectious agent is administered to or blood exposed to is a scavenger that feeds on carrion, such as crows, vultures, condors, hawks, eagles, hyenas, jackals, leopards, lions, opossums, raccoons, ravens, seagulls, lobsters, crabs, sharks, Tasmanian devils, bears, coyotes, wolves, Komodo dragons, crocodiles, and alligators. FIG. 1 illustrates an implementation of the method of the disclosure where the non-domesticated wild animal is a hippopotamus. FIG. 2 illustrates an implementation of the method of the disclosure where the non-domesticated wild animal is a crocodile. The figures feature particular implementations where a virus isolated from a human patient is administered directly to the hippo by injection (FIG. 1) or to blood taken from the crocodile (FIG. 2). Serum samples are taken from the blood from the hippo or crocodile after immunization and one or more immunotherapeutic infectious disease fighting components (e.g., antiviral, antibacterial, antifungal, antihelminth, antiprotozoal, anti-prion), in the sera including but not limited to antibodies, cytokines such as interleukins, interferons, chemokines, lymphokines, monokines, tumor necrosis factors, transforming growth factors, and haematopoietins, components of the complement system, and acute phase proteins are isolated. The isolated immunotherapeutic component or components are administered to the human patient, such as by injection (e.g., intravenous), by inhalation through an aerosolized spray.

The bacteria, virus, or other infectious agent can be administered to the non-domesticated wild animal through routes of administration such as parenteral (e.g., subcutaneous, intradermal, intramuscular, intraperitoneal, intravenous). The non-domesticated wild animal can be temporarily tranquilized during administration of the bacterial, virus, or other infectious agent or during blood collection and restrained or held in captivity for a period of time until administration and blood collection is completed.

The other infectious agent isolated from the human patient can include a fungi or a parasite such as a helminth or protozoa. The other infectious agent can also include a prion.

The other immunotherapeutic disease fighting components (other than antibodies) isolated from the blood of the non-domesticated wild animal can include other soluble molecules of the immune system of the non-domesticated wild animal including immunoproteins, immunopolypeptides, and immunopeptides, such as cytokines including interleukins, interferons, chemokines, lymphokines, monokines, tumor necrosis factors, transforming growth factors, and haematopoietins, components of the complement system, and acute phase proteins.

Methods of isolation of the bacteria, virus, or other infectious agent from a human patient can include techniques used for the specific type of infection. For example, for a respiratory infection, such techniques include swabbing an oropharyngeal or nasopharyngeal sample from an infected patient, inoculated cell cultures such as Vero cells with the sample to grow the infectious agent, determining cytopathic effects of the infectious agent with the cell cultures, and observing the infectious agent under microscopy to determine the nature of the infectious agent. This technique can be modified for pathogens other than respiratory viruses such as obtaining the infectious agent from skin lesions caused by the infectious agent, such as from exudate, from blood samples, or from biopsies of infected tissue. Additional steps can include isolation and purification of the infectious agent from cell culture supernatants, extracting genomic DNA (or genomic RNA as the case may be) from the infectious agent, and genomic sequencing of the infectious agent. The genome of the infectious agent can be compared to that of known infectious agents of humans to determine whether the infectious agent is novel or recent as an infectious agent to humans, based on the genomic sequencing information. Alternatively or in addition, protein can be extracted from the infectious agent and sequenced and its protein sequence compared to that of known infectious agents in a similar manner.

The bacteria, virus, or other infectious agent can be one that has never infected humans. The pandemic event can originate from a zoonotic event, through accidental release from a laboratory, or through intentional exposure of humans (e.g., bioterrorism, biological warfare). As such, the bacteria, virus, or other infectious agent can be one for which there is no standard or conventional treatment.

Methods of exposing the non-domesticated wild animal to or inoculating with the bacteria, virus, or other infectious agent can include administration by oral ingestion, inhalation of aerosolized agent through the mouth or nasal cavity, or parenteral administration. Such methods are useful for generating polyclonal antibodies. Polyclonal antibodies immunospecific for the bacteria, virus, or other infectious agent can be produced by various procedures well-known in the art, such as those described by the references cited above. For example, the bacteria, virus, or other infectious agent can be administered to one or more non-domesticated wild animals described herein as host animals, to induce the production of sera containing polyclonal antibodies specific for the bacteria, virus, or other infectious agent. Various adjuvants may be used to increase the immunological response, including but are not limited to, Freund's (complete and incomplete), mineral gels such as aluminum hydroxide, surface active substances such as lysolecithin, pluronic polyols, polyanions, peptides, oil emulsions, keyhole limpet hemocyanins, dinitrophenol, and potentially useful human adjuvants such as BCG (bacille Calmette-Guerin) and Corynebacterium parvum.

Sera can be collected immediately before immunization with the infectious agent. Immunization of non-domesticated wild animals can occur multiple times (i.e., prime administration plus booster doses). Immune blood samples can be collected after last immunization at select periods (e.g., daily, semiweekly, or weekly) over a period of several weeks post infection to allow an immune response in the non-domesticated wild animal to develop. Plasma and sera can be obtained from the immune blood samples and immunoglobulin can be prepared from the sera using known methods. The sera or immunoglobulin preparation can be tested using a microtiter neutralization plaque assay to measure efficacy of the sera or immunoglobulin preparation for protecting against cytotoxicity by the infectious agent in vitro. Examples of cell lines used in plaque assays include Vero and MDBK and can be chosen according to the characterization of the infectious agent isolated from the human patient. Such assay involves serial dilution of the sera or immunoglobulin preparations from the non-domesticated wild animal in microtiter plates to determine plaque reduction neutralizing titers from the tested sera. The plaque reduction neutralization titres can be expressed as the reciprocal of the highest dilution that gave 50% plaque reduction. Peak antibody production from the non-domesticated wild animal can be determined from the measured plaque reduction neutralization titres.

Antibodies can be purified from the effective sera samples as follows. Sera can be collected from the non-domesticated wild animals at peak antibody production after inoculation with the infectious agent and irradiated to eliminate any residual virus present in the sera (in the event the infectious agent is determined to be a virus); samples can be examined by polymerase chain reaction (PCR) to ensure that the sera is virus free. The antibody fraction of the sera can be purified by density centrifugation, dialyzed to remove gradient, and concentrated. Purity can be established using RT-PCR analysis. Neutralization activity of the purified antibodies can be confirmed using the microtiter plaque neutralization assay. Other soluble immunotherapeutic agents such as various cytokines (e.g., interleukins, interferons, chemokines, lymphokines, monokines, tumor necrosis factors, transforming growth factors, and haematopoietins), components of the complement system, and acute phase proteins can be isolated from sera of inoculated non-domesticated wild animals as well, or from cultured blood obtained from the non-domesticated wild animals and exposed to the bacteria, virus, or other infectious agent in vitro. These can be identified using known analytical techniques that compare serum or sera obtained from the non-domesticated wild animal or animals before and after inoculation of the animal(s), or by comparing serum or sera from blood taken from the non-domesticated wild animal(s) and exposed to the bacteria, virus, or other infectious agent in vitro to serum or sera from non-exposed blood taken from the non-domesticated wild animal(s). Such analytical techniques can include protein purification, two-dimensional gel electrophoresis, western blotting, high-performance liquid chromatography, thin-layer chromatography, gas chromatography, mass spectrometry, and protein or peptide sequencing. Details for such analytical techniques are available in references cited above.

The infectious disease fighting components or immune effectors identified by said analytical techniques can be of types or families of infectious disease fighting components previously characterized in animals and humans. Such immune effectors are typically proteins (immunoproteins), polypeptides (immunopolypeptides) or peptides (immunopeptides). The infectious disease fighting components or immune effectors can include those that act directly to neutralize the virus, bacteria, fungus, helminth, protozoa, or prion causing the infectious disease, such as antibodies, or can include components that stimulate other components of the immune system such as immune cells (e.g., CD4+ T cells, CD8+ T Cells, B cells, macrophages) to fight the infection, such components including cytokines such as interleukins and interferons. Chemokines are chemoattractant cytokines that influence migration of immune cells. The complement system has multiple functions including inducing inflammation and opsonization of pathogens. Acute phase proteins have a diverse repertoire of functions in the regulation of immunity and inflammation. These are merely brief overviews of the functions of a representation of the major classes and families of immune effectors and those of skill in the art will understand their full ranges of members and functions. The infectious disease fighting components or immune effectors identified by said analytical techniques can also include immunoproteins, immunopolypeptides, immunopeptides not previously characterized, or can be primarily chemical in nature.

Such isolated and purified antibodies obtained from the non-domesticated wild animal or animals are polyclonal and can be used directly to treat the human patient. In other implementations, monoclonal antibodies can be produced using non-domesticated wild animals such as wild mice, wild rabbits, wild rats, or other non-domesticated wild animals such as others recited in this disclosure as host species. The monoclonal antibodies can be prepared using a wide variety of techniques known in the art, such as those described by the references cited above and hereafter, including the use of hybridoma, recombinant, and phage display technologies, or a combination thereof. For example, monoclonal antibodies can be produced using hybridoma techniques, including those known in the art and taught, for example, in Harlow et al., Antibodies: A Laboratory Manual, (Cold Spring Harbor Laboratory Press, 2nd ed. 1988); and Hammerling et al., in: Monoclonal Antibodies and T Cell Hybridomas 563 681 (Elsevier, N.Y., 1981). The term “monoclonal antibody” as used herein is not limited to antibodies produced through hybridoma technology. The term “monoclonal antibody” refers to an antibody that is derived from a single clone, including any eukaryotic, prokaryotic, or phage clone, and not the method by which it is produced.

Methods for producing and screening for specific antibodies using hybridoma technology are routine and well known in the art. Briefly, the non-domesticated wild host animal can be immunized with the bacteria, virus, or other infectious agent obtained from the human patient, and once an immune response is detected, e.g., antibodies specific for the antigen or antigens are detected in the host animal's serum, the host animal's spleen is harvested and splenocytes isolated. The splenocytes are then fused by well-known techniques to any suitable myeloma cells, for example cells from cell line SP20 available from the ATCC. Hybridomas are selected and cloned by limited dilution. The hybridoma clones are then assayed by methods known in the art for cells that secrete antibodies capable of binding the bacteria, virus, or other infectious agent. Ascites fluid, which generally contains high levels of antibodies, can be generated by immunizing the host animal with positive hybridoma clones.

The antibodies can also be engineered in a laboratory into structures that enhance their efficacy and stability. Such structures include antibody fragments such as Fabs (F(ab) fragments, F(ab′) fragments, F(ab′)₂ fragments). Fabs may be produced by proteolytic cleavage of immunoglobulin molecules, using enzymes such as papain (to produce Fab fragments) or pepsin (to produce F(ab′)2 fragments). F(ab′)2 fragments contain the variable region, the light chain constant region and the CH1 domain of the heavy chain. Other antibody structures that can be derived from the isolated antibodies include single-chain Fv fragments (scFv).

In phage display methods, functional antibody domains are displayed on the surface of phage particles which carry the polynucleotide sequences encoding them. In particular, DNA sequences encoding VH and VL domains are amplified from animal cDNA libraries. The DNA encoding the VH and VL domains are recombined together with a scFv linker by PCR and cloned into a phagemid vector. The vector is electroporated in E. coli, and the E. coli is infected with helper phage. Phage used in these methods are typically filamentous phage including fd and M13 and the VH and VL domains are usually recombinantly fused to either the phage gene III or gene VIII. Phage expressing an antigen binding domain that binds to a particular antigen can be selected or identified with antigen, e.g., using labeled antigen or antigen bound or captured to a solid surface or bead. Examples of phage display methods that can be used to make the antibodies of the present disclosure include those disclosed in Brinkman et al., 1995, J. Immunol. Methods 182:41-50; Ames et al., 1995, J. Immunol. Methods 184: 177-186; Kettleborough et al., 1994, Eur. J. Immunol. 24:952-958; Persic et al., 1997, Gene 187:9-18; Burton et al., 1994, Advances in Immunology 57:191-280; International application No. PCT/GB91/01134; International publication Nos. WO 90/02809; WO 91/10737; WO 92/01047; WO 92/18619; WO 93/11236; WO 95/15982; WO 95/20401; and WO97/13844; and U.S. Pat. Nos. 5,698,426; 5,223,409; 5,403,484; 5,580,717; 5,427,908; 5,750,753; 5,821,047; 5,571,698; 5,427,908; 5,516,637; 5,780,225; 5,658,727; 5,733,743; and 5,969,108.

As described in the above references, after phage selection, the antibody coding regions from the phage can be isolated and used to generate whole antibodies or any other desired antigen binding fragment, and expressed in any desired host, including mammalian cells, insect cells, plant cells, yeast, and bacteria, e.g., as described below. Techniques to produce Fab, Fab′ and F(ab′)2 fragments recombinantly can also be employed using methods known in the art such as those disclosed in PCT publication No. WO 92/22324; Mullinax et al., 1992, BioTechniques 12(6):864-869; Sawai et al., 1995, AJRI 34:26-34; and Better et al., 1988, Science 240:1041-1043.

The antibodies or their fragments can be prepared by chemical synthesis or by recombinant DNA engineering techniques (see, e.g., U.S. Publication No. 2005/0084449, which is incorporated herein in its entirety) and expressed in cells in culture such as mammalian cell expression systems or in other systems such as plants.

To generate whole antibodies, PCR primers including VH or VL nucleotide sequences, a restriction site, and a flanking sequence to protect the restriction site can be used to amplify the VH or VL sequences in scFv clones. Utilizing cloning techniques known to those of skill in the art, the PCR amplified VH domains can be cloned into vectors expressing a VH constant region, e.g., the human gamma 4 constant region, and the PCR amplified VL domains can be cloned into vectors expressing a VL constant region, e.g., human kappa or lambda constant regions. Preferably, the vectors for expressing the VH or VL domains comprise an EF-1α promoter, a secretion signal, a cloning site for the variable domain, constant domains, and a selection marker such as neomycin. The VH and VL domains may also be cloned into one vector expressing the necessary constant regions. The heavy chain conversion vectors and light chain conversion vectors are then co-transfected into cell lines to generate stable or transient cell lines that express full-length antibodies, IgG, using techniques known to those of skill in the art.

The antibodies can also be engineered such that antigen binding sites of the antibodies obtained from the non-domesticated wild animal can be inserted into a human antibody scaffold resulting in an antibody with decreased immunogenicity in humans compared to the antibodies from the non-domesticated wild animal. Such engineered antibodies can include chimeric antibodies where entire antigen-binding sites from antibodies obtained from the immunized non-domesticated wild animal or animals are inserted, or humanized antibodies where the complementarity-determining regions (CDRs) are inserted. A chimeric antibody is a molecule in which different portions of the antibody are derived from different immunoglobulin molecules. Methods for producing chimeric antibodies are known in the art. See e.g., Morrison, 1985, Science 229:1202; Oi et al., 1986, BioTechniques 4:214; Gillies et al., 1989, J. Immunol. Methods 125:191-202; and U.S. Pat. Nos. 5,807,715; 4,816,567; 4,816,397; and 6,311,415. A humanized antibody is an antibody or its variant or fragment thereof which is capable of binding to a predetermined antigen and which comprises a framework region having substantially the amino acid sequence of a human immunoglobulin and a CDR having substantially the amino acid sequence of a non-human immunoglobulin, such as an immunoglobin from an inoculated non-domesticated wild animal. A humanized antibody comprises substantially all of at least one, and typically two, variable domains (Fab, Fab′, F(ab′)2, Fabc, Fv) in which all or substantially all of the CDR regions correspond to those of a non-human immunoglobulin (i.e., donor antibody) and all or substantially all of the framework regions are those of a human immunoglobulin consensus sequence. Preferably, a humanized antibody also comprises at least a portion of an immunoglobulin constant region, typically that of a human immunoglobulin. Ordinarily, the antibody will contain both the light chain as well as at least the variable domain of a heavy chain. The antibody also may include the CH1, hinge, CH2, CH3, and CH4 regions of the heavy chain. The humanized antibody can be selected from any class of immunoglobulins, including IgM, IgG, IgD, IgA and IgE, and any isotype, including IgG1, IgG2, IgG3 and IgG4. Usually the constant domain is a complement fixing constant domain where it is desired that the humanized antibody exhibits cytotoxic activity, and the class is typically IgG1. Where such cytotoxic activity is not desirable, the constant domain may be of the IgG2 class. The humanized antibody may comprise sequences from more than one class or isotype, and selecting particular constant domains to optimize desired effector functions is within the ordinary skill in the art. The framework and CDR regions of a humanized antibody need not correspond precisely to the parental sequences, e.g., the donor CDR or the consensus framework may be mutagenized by substitution, insertion or deletion of at least one residue so that the CDR or framework residue at that site does not correspond to either the consensus or the import antibody. Such mutations, however, will not be extensive. Usually, at least 75% of the humanized antibody residues will correspond to those of the parental framework region (FR) and CDR sequences, more often 90%, and most preferably greater than 95%. Humanized antibody can be produced using variety of techniques known in the art, including but not limited to, CDR grafting (European Patent No. EP 239,400; International Publication No. WO 91/09967; and U.S. Pat. Nos. 5,225,539; 5,530,101; and 5,585,089), veneering or resurfacing (European Patent Nos. EP 592,106 and EP 519,596; Padlan, 1991, Molecular Immunology 28(415):489 498; Studnicka et al., 1994, Protein Engineering 7(6):805 814; and Roguska et al., 1994, PNAS 91:969 973), chain shuffling (U.S. Pat. No. 5,565,332), and techniques disclosed in, e.g., U.S. Pat. Nos. 6,407,213; 5,766,886; WO 9317105; Tan et al., J. Immunol. 169:1119-25 (2002); Caldas et al., Protein Eng. 13(5):353-60 (2000); Morea et al., Methods 20(3):267-79 (2000); Baca et al., J. Biol. Chem. 272(16):10678-84 (1997); Roguska et al., Protein Eng. 9(10): 895-904 (1996); Couto et al., Cancer Res. 55 (23 Supp):5973s-5977s (1995); Couto et al., Cancer Res. 55(8): 1717-22 (1995); Sandhu J S, Gene 150(2):409-10 (1994); and Pedersen et al., J. Mol. Biol. 235(3): 959-73 (1994). Often, framework residues in the framework regions will be substituted with the corresponding residue from the CDR donor antibody to alter, preferably improve, antigen binding. These framework substitutions are identified by methods well known in the art, e.g., by modeling of the interactions of the CDR and framework residues to identify framework residues important for antigen binding and sequence comparison to identify unusual framework residues at particular positions. (See, e.g., Queen et al., U.S. Pat. No. 5,585,089; and Riechmann et al., 1988, Nature 332:323).

The disclosure includes purification of the isolated antibodies obtained from the non-domesticated wild animal, their engineered components, or other soluble effectors of the immune system including the various cytokines, components of the complement system, and acute phase proteins to an extent that other soluble non-therapeutic components obtained from the blood or sera of the non-domesticated wild animal that may cause adverse reactions such as blood clotting or inflammation are eliminated or reduced to an extent that they are at a concentration below the no-observed-adverse-effect level in humans for that particular soluble non-therapeutic component. For example, monoclonal antibodies may be purified by Protein-A chromatography. Other chromatography purification methods can be chosen based on the properties of the particular protein or chemical effector, the methods including affinity chromatography, ion exchange chromatography, size exclusion chromatography, hydrophobic interaction chromatography, and reversed phase chromatography.

The isolated and purified monoclonal antibodies, polyclonal antibodies, or antibody fragments specific to antigens on the bacteria, virus, or other infectious agent used to immunize the non-domesticated wild animal can be used in immunoassays to measure the amount of antigen or antigens or used in immunoaffinity purification.

The isolated and purified antibodies obtained from the non-domesticated wild animal or animals or their blood, their engineered components, or other soluble effectors of the immune system of the non-domesticated wild animal such as the various cytokines (e.g., interleukins, interferons, chemokines, lymphokines, monokines, tumor necrosis factors, transforming growth factors, and haematopoietins), components of the complement system, and acute phase proteins can be biomanufactured from living cells in a biologics facility in a bioreactor using production cell lines such as E. coli bacterial cells or Chinese hamster ovary cells, or can be synthesized chemically in process reactors in a pharmaceutical manufacturing facility.

The isolated and purified antibodies obtained from the non-domesticated wild animal or animals or their blood, their engineered components, or other soluble proteinaceous effectors of the immune system of the non-domesticated wild animal such as the various cytokines, components of the complement system, and acute phase proteins can be engineered to increase stability, increase efficacy, increase solubility, increase protease resistance, reduce immunogenicity, or improve pharmacokinetic parameters such as increase Tmax or increase bioavailability. Known engineering methods include covalently or non-covalently linking effectors such as immunoproteins to albumin, transferrin or polyethylene glycol (PEG). Other known methods of protein modification to improve the biopharmaceutical properties of proteins include site-selective chemical modification such N-terminal acylation and a C-terminal amidation, addition of an N-terminal cysteine, and cyclization.

The isolated and purified antibodies obtained from the non-domesticated wild animal, their engineered components, or other soluble effectors of the immune system of the non-domesticated wild animal such as the various cytokines, components of the complement system, and acute phase proteins can be administered directly to the human patient or patients suffering from the infectious disease to treat the disease. Such isolated immunotherapeutic agents can be administered as a combination of agents isolated directly from the non-domesticated wild animal or animals including its serum or sera or blood or as one or more individual components from stock manufactured in a biologics or pharmaceutical manufactured facility for a large scale production of therapeutic biologics or pharmaceuticals to be administered to patient populations suffering from the infectious disease. Route of administration of the isolated and purified antibodies, their engineered components, or other soluble effectors of the immune system to the human patient can be intravenous by bolus dose or infusion, or other parenteral routes including but not limited to subcutaneous, intradermal, intramuscular, intraperitoneal, intraarterial, and intrathecal. Parenteral formulations can include isotonic drug diluents such as 0.9% NaCl, 5% dextrose in water, and Ringer's lactate solution. Aerosol sprays suitable for inhalation by the patient are also contemplated. Various formulations of both biologics and pharmaceuticals are discussed in Remington cited above.

The immunotherapeutic agents including antibodies, and the various cytokines, components of the complement system, and acute phase proteins can be administered the human patient suffering from the infectious disease according to established principles in the pharmaceutical sciences. In one implementation, the immunotherapeutic agent or agents are administered by bolus IV injection one daily or twice daily. In other implementations, the immunotherapeutic agent or agents are administered by infusion. In other implementations, the immunotherapeutic agent or agents are administered by inhalation. In other implementations, the immunotherapeutic agent or agents are administered orally. The immunotherapeutic agent or agents are administered for a period of time such as one day, two days, three days, four days, five days, six days, 1 week, 2 weeks, 3 weeks, 4 weeks, until the human patient's symptoms improve and the patient recovers from the infectious disease.

The disclosure includes administration of additional agents with the isolated and purified antibodies, their engineered components, or other soluble effectors of the immune system to assist in the healing process such as vitamins, medicines, supplements, other antibodies, enzymes to prevent blood clots, or antivirals or antibiotics. Such agents can be formulated with the purified antibodies, their engineered components, or other soluble immunotherapeutic effectors in the parenteral formulations or spray solutions, or can be formulated and administered separately.

Side effects of the immunotherapeutic methods of the disclosure are expected to be similar to existing immunotherapy such as antibody therapy for infectious diseases or for other disease such as cancer, including reactions at the injection site of the human patient such as redness, soreness, or swelling, and flu-like symptoms such as chills, fever, and myalgia. Rare allergic reactions are also possible but can be mitigated by therapeutics such as epinephrine.

The methods of the disclosure are useful for treating infections in human patients, particularly when the infectious agent has only recently infected humans and existing treatments are ineffective. The immunoproteins, immunopolypeptides, and immunopeptides isolated from the methods of the disclosure are also useful as research reagents.

The present disclosure has described particular implementations having various features. In light of the disclosure provided above, it will be apparent to those skilled in the art that various modifications and variations can be made without departing from the scope or spirit of the disclosure. One skilled in the art will recognize that the disclosed features may be used singularly, in any combination, or omitted based on the requirements and specifications of a given application or design. When an implementation refers to “comprising” certain features, it is to be understood that the implementations can alternatively “consist of” or “consist essentially of” any one or more of the features. Other implementations will be apparent to those skilled in the art from consideration of the specification and practice of the disclosure.

It is noted in particular that where a range of values is provided in this specification, each value between the upper and lower limits of that range is also specifically disclosed. The upper and lower limits of these smaller ranges may independently be included or excluded in the range as well. The singular forms “a,” “an,” and “the” include plural referents unless the context clearly dictates otherwise. It is intended that the specification and examples be considered as exemplary in nature and that variations that do not depart from the essence of the disclosure fall within the scope of the disclosure. Further, all of the references cited in this disclosure including patents, published applications, and non-patent literature are each individually incorporated by reference herein in their entireties and as such are intended to provide an efficient way of supplementing the enabling disclosure as well as provide background detailing the level of ordinary skill in the art.

1. A method, comprising:
isolating an infectious agent from a human patient infected with the infectious agent;
obtaining one or more immunoproteins, immunopolypeptides, and immunopeptides from serum from a non-domesticated wild animal by:
inoculating, in vivo, the non-domesticated wild animal with the isolated infectious agent or inoculating, in vitro, blood taken from the non-domesticated wild animal with the isolated infectious agent;
comparing a protein, polypeptide, and peptide composition of serum obtained from blood from the non-domesticated wild animal inoculated in vivo or serum obtained from in vitro inoculated blood from the non-domesticated wild animal to that of a serum sample obtained from blood from the non-domesticated wild animal prior to inoculation or from non-inoculated blood, to identify the one or more immunoproteins, immunopolypeptides, and immunopeptides; and
isolating and purifying the one or more immunoproteins, immunopolypeptides, and immunopeptides; and
administering the one or more immunoproteins, immunopolypeptides, and immunopeptides to the human patient infected with the infectious agent.

2. The method of claim 1, wherein the infectious agent is a virus, bacteria, fungus, helminth, protozoa, or prion.

3. The method of claim 1, wherein the one or more immunoproteins, immunopolypeptides, and immunopeptides are chosen from antibodies, cytokines, components of the complement system, and acute phase proteins.

4. The method of claim 1, further comprising manufacturing the one or more immunoproteins, immunopolypeptides, and immunopeptides in a biologics facility before administration to the human patient.

5. The method of claim 4, further comprising engineering the one or more immunoproteins, immunopolypeptides, and immunopeptides during manufacturing to increase stability, increase efficacy, increase solubility, increase protease resistance, reduce immunogenicity, and/or improve pharmacokinetic parameters of the immunoproteins, immunopolypeptides, and immunopeptides in humans.

6. The method of claim 1, wherein the non-domesticated wild animal is a hippopotamus, snow leopard, amur leopard, puma, dogfish shark, dolphin, zebra, buffalo, ostrich, elephant, alligator, crocodile, hyena, or bear.

7. The method of claim 1, wherein the non-domesticated wild animal is a scavenger.

8. The method of claim 7, wherein the scavenger is a crow.

9. The method of claim 7, wherein the scavenger is a vulture.

10. The method of claim 7, wherein the scavenger is a condor.

11. The method of claim 7, wherein the scavenger is a hawk.

12. The method of claim 7, wherein the scavenger is an eagle.

13. The method of claim 7, wherein the scavenger is a hyena.

14. The method of claim 7, wherein the scavenger is a jackal.

15. The method of claim 7, wherein the scavenger is a leopard.

16. The method of claim 7, wherein the scavenger is a lion.

17. The method of claim 7, wherein the scavenger is an opossum.

18. The method of claim 7, wherein the scavenger is a raccoon.

19. The method of claim 7, wherein the scavenger is a raven.

20. The method of claim 7, wherein the scavenger is a seagull.
